(19) United States
(12) Patent Application Publication
(10) Pub. No.: US 2023/0233813 A1
(43) Pub. Date: Jul. 27, 2023

(54) INTRODUCER NEEDLE HAVING A FLASHBACK NOTCH AND RELATED DEVICES

(71) Applicant: Becton, Dickinson and Company, Franklin Lakes, NJ (US)

(72) Inventors: Xiwei Chen, Shanghai (CN); Linda Kunardi, Singapore (SG); Xue Guang John Ong, Shanghai (CN); Bo Yan, Shanghai (CN); Gary Liu, Shanghai (CN)

(21) Appl. No.: 18/075,220

(22) Filed: Dec. 5, 2022

Related U.S. Application Data
(60) Provisional application No. 63/302,898, filed on Jan. 25, 2022.

(51) Int. Cl. A61M 25/06

(52) U.S. Cl. CPC A61M 25/0693; A61M 25/0618; A61M 25/0606

(57) ABSTRACT

An intravenous (IV) catheter assembly may include a catheter adapter and a catheter extending from the distal end of the catheter adapter. The intravenous (IV) catheter assembly may include an introducer needle. The elongated shaft may include a flashback notch, and the flashback notch may include a distal end and a proximal end. The flashback notch may include a smooth tapered edge extending from the distal end in a direction that increases a depth of the flashback notch. The smooth tapered edge may extend along at least half of an entire length of the flashback notch between the distal end and the proximal end. In an orthogonal side view projection of the intravenous catheter assembly, every tangent to the smooth tapered edge may be at an angle of 30° or less with respect to a longitudinal axis of the introducer needle

### RELATED APPLICATIONS

This application claims the benefit of U.S. Provisional Patent Application No. 63/302,898, filed Jan. 25, 2022, and entitled INTRODUCER NEEDLE HAVING A FLASHBACK NOTCH AND RELATED DEVICES, which is incorporated herein in its entirety.

### BACKGROUND

A common type of catheter assembly includes a peripheral intravenous catheter (“PIVC”) that is over-the-needle. As its name implies, the PIVC that is over-the-needle may be mounted over an introducer needle having a sharp distal tip. The catheter assembly may include a catheter adapter, the PIVC extending distally from the catheter adapter, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter assembly. In some instances, blood may travel into the introducer needle and then out of a flashback notch in the introducer needle to reach the flashback chamber, where the blood is visible to the clinician. Once placement of the introducer needle has been confirmed by observation of the blood, the clinician may remove the introducer needle, leaving the PIVC in place in the blood vessel for future blood withdrawal or fluid infusion.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices. More particularly, in some embodiments, the present disclosure relates to an intravenous (IV) catheter assembly. In some embodiments, the IV catheter assembly may include a catheter adapter, which may include a distal end, a proximal end, a lumen extending through the distal end of the catheter adapter and the proximal end of the catheter adapter, and an inner wall forming the lumen. In some embodiments, the IV catheter assembly may include a catheter extending from the distal end of the catheter adapter. In some embodiments, the catheter may include a peripheral IV catheter, a midline catheter, or a peripherally-inserted central catheter.

In some embodiments, the IV catheter assembly may include an introducer needle, which may include a sharp distal tip and an elongated shaft extending from the sharp distal tip. In some embodiments, the introducer needle may be movable between a ready position in which the sharp distal tip is distal to a distal end of the catheter and a retracted position proximal to the ready position. In some embodiments, the elongated shaft may include a flashback notch. In some embodiments, the flashback notch may include a distal end and a proximal end. In some embodiments, the flashback notch may include a smooth tapered edge extending from the distal end in a direction that increases a depth of the flashback notch. In some embodiments, the smooth tapered edge may extend along at least half of an entire length of the flashback notch between the distal end and the proximal end.

In some embodiments, the IV catheter assembly may include a needle guard slidably mounted on the introducer needle. In some embodiments, a portion of the needle guard may abut the inner wall of the catheter adapter to retain the needle guard in the catheter adapter when the introducer needle is in the ready position. In some embodiments, in response to movement of the introducer needle from the ready position to the retracted position, the portion of the needle guard may be released from the inner wall and block the sharp distal tip.

In some embodiments, a width of the flashback notch may increase from the distal end to the proximal end to form a tear-drop shape. In some embodiments, the flashback notch may include a step extending from the proximal end of the flashback notch. In some embodiments, the step may be transverse to a longitudinal axis of the introducer needle. In some embodiments, the smooth tapered edge may extend adjacent the step. In some embodiments, the flashback notch may have an oval shape. In some embodiments, in an orthogonal side view projection of the IV catheter assembly, every tangent to the smooth tapered edge may be at an angle of 30° or less with respect to the longitudinal axis of the introducer needle. In these and other embodiments, in the orthogonal side view projection of the IV catheter assembly, every tangent to the smooth tapered edge may be at an angle greater than or equal to 10° with respect to the longitudinal axis of the introducer needle.

In some embodiments, the introducer needle further may include a needle feature disposed on the elongated shaft. In some embodiments, the needle feature may include a first indent and a second indent opposite the first indent. In some embodiments, the first indent may be aligned with the flashback notch. In some embodiments, the needle feature may include a first protrusion and a second protrusion opposite the first protrusion. In some embodiments, the first protrusion and the second protrusion may be disposed between the first indent and the second indent.

In some embodiments, the first indent may be disposed on a top of the introducer needle, and the second indent is disposed on a bottom of the introducer needle. In some embodiments, the first protrusion and the second protrusion may be disposed on opposing sides of the introducer needle. In some embodiments, in response to movement of the introducer needle from the ready position to the retracted position, the elongated shaft may no longer exert a force on the portion such that the portion is released from the inner wall and blocks the sharp distal tip.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is a cross-sectional view of a prior art catheter assembly, illustrating a prior art introducer needle in a ready position;

FIG. 1B is a side view of an introducer needle of the prior art catheter assembly in a retracted position within a needle guard;

FIG. 1C is an upper perspective view of the needle guard of the prior art catheter assembly;

FIG. 1D is another upper perspective view of the needle guard of the prior art catheter assembly;

FIG. 2A is a top view of an example introducer needle;

FIG. 2B is a side view of the introducer needle of FIG. 2A;

FIG. 2C is a side view of the introducer needle of FIG. 2A being retracted proximally with respect to the prior art needle guard;

FIG. 3A is a top view of an introducer needle, illustrating an example flashback notch having a tear-drop shape, according to some embodiments;

FIG. 3B is a side view of the introducer needle, illustrating the flashback notch having the tear-drop shape, according to some embodiments;

FIG. 3C is a cross-sectional view of an example catheter assembly, according to some embodiments;

FIG. 3D is an enlarged orthogonal side view projection of the IV catheter assembly having the tear-drop shape, according to some embodiments;

FIG. 4A is a top view of the introducer needle, illustrating the flashback notch having an oval shape, according to some embodiments; and

FIG. 4B is a side view of the introducer needle, illustrating the flashback notch having the oval shape, according to some embodiments.

### DESCRIPTION OF EMBODIMENTS

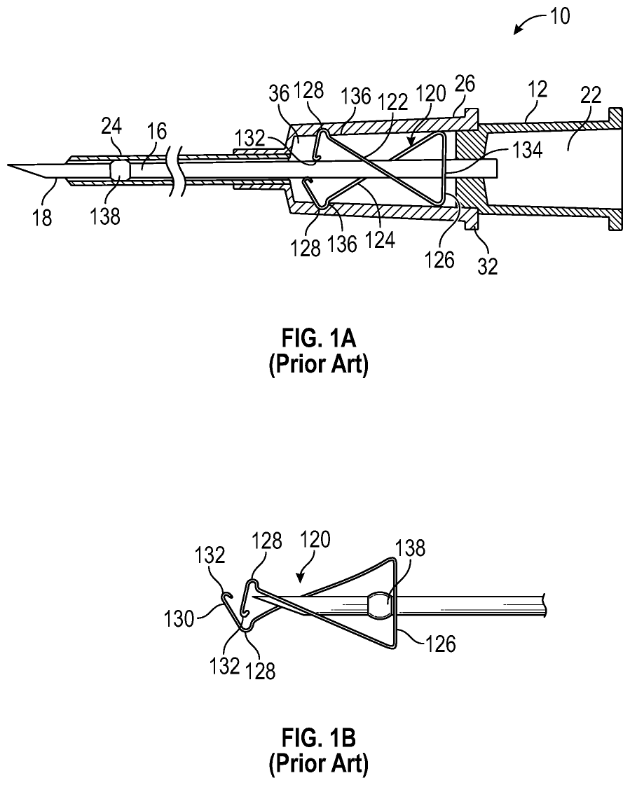

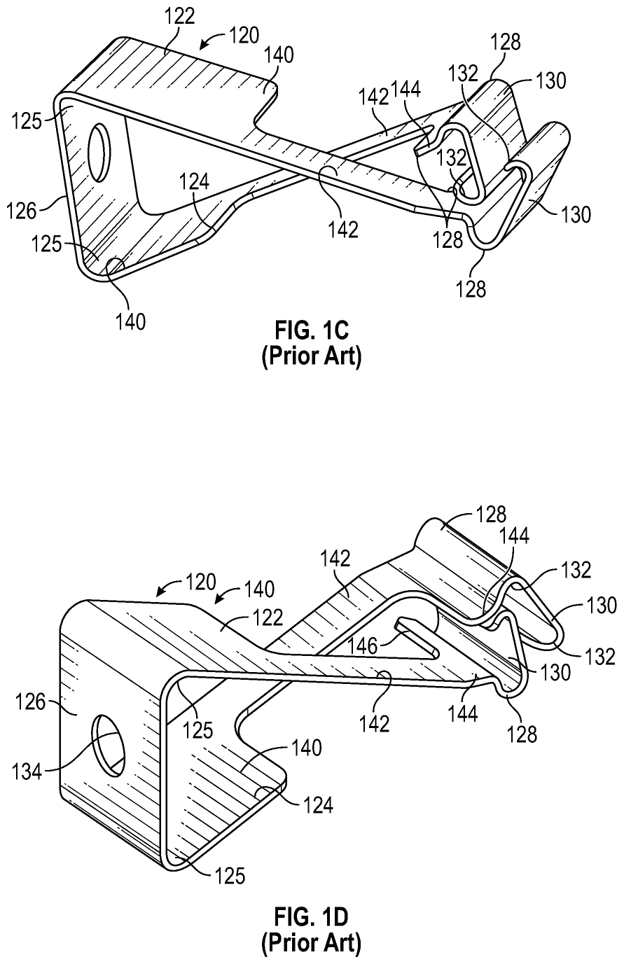

Referring now to FIGS. 1A-1D, a prior art catheter assembly 10 includes a needle hub 12 that includes an axial opening. The axial opening securely receives a proximal end of an introducer needle 16 having a sharp tip 18. The needle hub 12, as is conventional, is hollow and includes a flash chamber 22. As is also conventional, the introducer needle 16 is received within a catheter 24, a proximal end of which is concentrically affixed within the distal end of a catheter adapter 26. The catheter adapter 26 terminates at its proximal end in a Luer fitting 32 adapted to receive a tubing set which, in a known manner, administers intravenous fluid into the patient.

In a ready position of the catheter assembly, illustrated in FIG. 1A, the distal end of the needle hub 12 is snugly received in the proximal end of the catheter adapter 26 such that the introducer needle 16 distally beyond the catheter adapter 26 and the catheter 24, so that the sharp tip 18 of the introducer needle 16 extends beyond a tapered distal end of the catheter 24.

In use, the sharp tip 18 of the introducer needle 16 and the catheter 24 are inserted into a patient's vein. Thereafter, a clinician manually places the catheter 24 further into the vein and then withdraws the introducer needle 16 by grasping and moving by hand the proximal end of the needle hub 12. The Luer fitting 32 of the catheter adapter 26 is then fitted with a source of the fluid that is to be administered into the patient's vein.

As the introducer needle 16 is being withdrawn from the patient, a needle guard 120 located within a hub chamber 36 automatically snaps into a retracted position in which the needle guard 40 blocks access to the sharp tip 18 and prevents further distal movement of the sharp tip 18, thereby to prevent accidental contact by the clinician with the sharp tip 18. The needle guard 120 includes first and second arms 122 and 124, respectively, joined at their proximal ends in a hinged arrangement to the ends of a rear wall 126. The distal ends of the arms 122, 124 each include a curved protrusion 128 extending to a distal end wall 130, which, in turn, terminates in a lip 132. The rear wall 126 includes an opening 134, and the arms 122 and 124 each include a narrow portion 142 that extends between a distal wide portion 140 and a proximal wide portion 144. A lateral clamping edge 146 is defined at the distal wide portion 140.

When the needle guard 120 is in its ready position, the curved protrusions 128 in each of the arms 122, 124 are received in an annular groove or ring 136 formed in the inner wall of the catheter adapter 26 which is removably fit into the distal end of the needle hub 12. The introducer needle 16 having the sharp tip 18 at its distal end is received within the catheter 24 which is secured to the distal end of the catheter adapter 26. The proximal end of the introducer needle 16 passes through the opening 134 in the rear wall 126. The introducer needle 16 includes a crimp 138 which is sufficiently small to allow the introducer needle 16 to move axially along the catheter 24 but is greater in width than the opening 134 for reasons to be described below.

In the ready position illustrated in FIG. 1A, a shaft of the introducer needle 16 passes through the needle guard 120 and applies an outward radial force on the resilient arms 122, 124 by means of its engagement with the lips 132, so as to urge the curved protrusions 128 of each of the arms into the annular groove 136, so as to retain the needle guard 120 in a fixed position within the inner wall of the catheter adapter 26. The shaft of the introducer needle 16 that passes through the needle guard 120 frictionally engages the inner edges of the narrow portion 142 of arms 122, 124 so as to further retain the needle in its ready position.

When the introducer needle 16 is retracted axially and proximally within the catheter adapter 26 and moves past the lip 132 of the needle guard, the radial force previously exerted on the arms 122, 124 of the needle guard 120 is suddenly released. This causes the distal end walls 130 of the needle guard to be released from their seat in the annular groove 136 and to pivot inward into the catheter adapter 26 until, as seen in FIGS. 1C-1D, the distal end walls 130 overlap one another at a location distally in front of the needle tip, thereby to form a barrier that prevents inadvertent contact with, and distal movement of, the sharp tip 18. At the same time, the clamping edges 146 of the needle guard 120 are urged against the sharp tip 18 to restrict further axial movement of the introducer needle 16.

As illustrated in FIG. 1B, the needle guard 120 and the introducer needle 16 clamped to the needle guard 120 after needle retraction can be removed from the catheter adapter 26 as a unitary assembly and safely discarded. If an attempt is made, intentionally or inadvertently, to pull the introducer needle 16 further proximally, as illustrated in FIG. 1B, out of the needle guard 120, the crimp 138 on the shaft will come into contact with the rear wall 126, and, since its width is greater than that of the opening 134, the rear wall 126 will at this point prevent any further axial movement of the introducer needle 16 out of the needle guard 120.

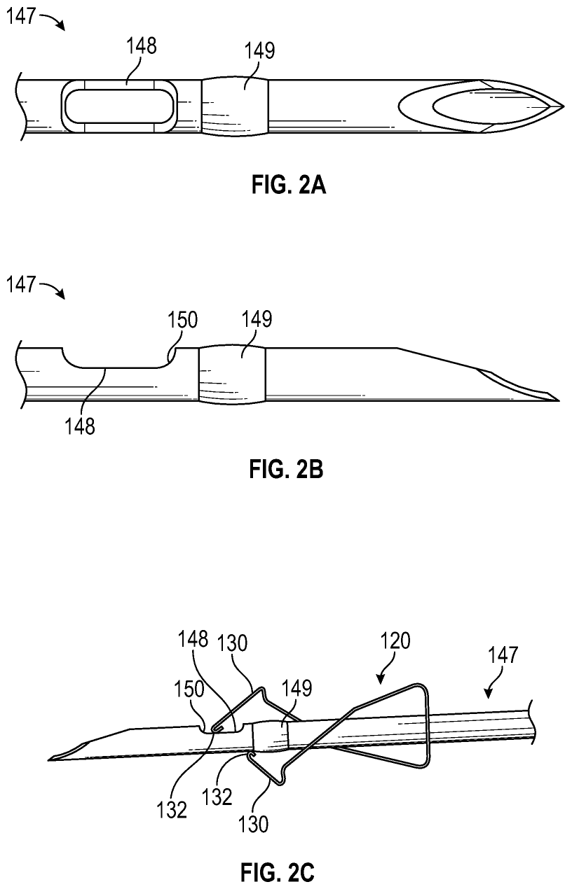

Referring now to FIGS. 2A-2C, in some embodiments, an introducer needle 147 may include a flashback notch 148, which may be distal to a needle feature 149 that may include an annular protrusion. In some embodiments, use of the introducer needle 147 with the needle guard 120 of the prior art catheter assembly 10 may result in the needle guard 120 getting caught on the flashback notch 148. In further detail, in some embodiments, when the introducer needle 147 is retracted axially and proximally within the catheter adapter 26, the distal end wall 130 and/or the lip 132 may abut the flashback notch 148 and get caught or hooked on the flashback notch 148, which may prevent smooth retraction of the introducer needle 147 and/or premature decoupling of the needle guard 120 from a catheter adapter. An example of the needle guard 120 caught on the flashback notch 148 is illustrated in FIG. 2C. In some embodiments, the needle guard 120 may get caught due to a steep incline 150 proximate a distal end of the flashback notch 148.

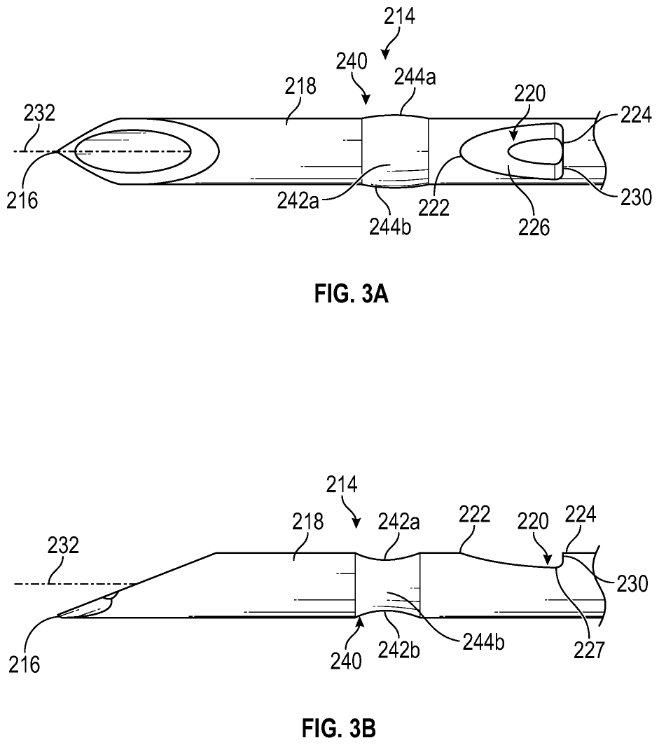

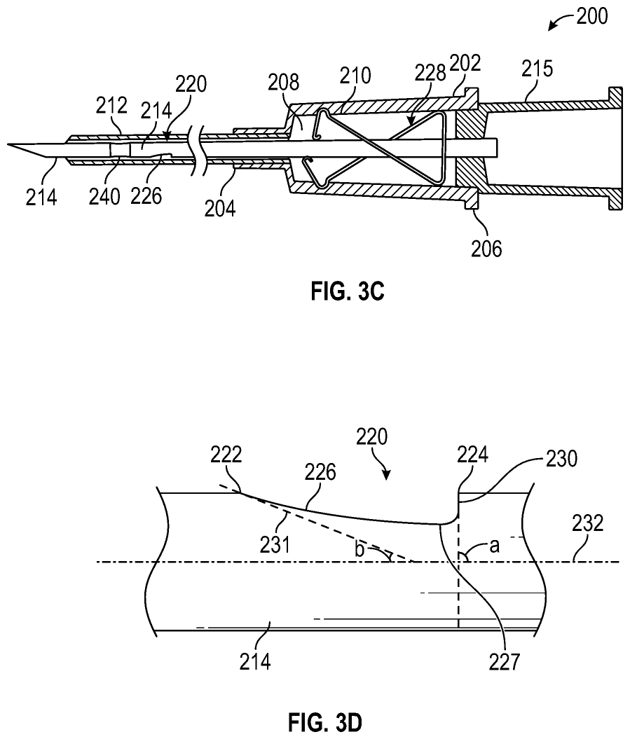

Referring now to FIGS. 3A-3D, an IV catheter assembly 200 is illustrated, according to some embodiments. In some embodiments, the IV catheter assembly 200 may be similar or identical to the prior art catheter assembly 10 in terms of one or more components and/or operation. In some embodiments, the IV catheter assembly 200 may include any suitable catheter assembly that includes an introducer needle with a flashback notch, such as, for example, the BD INSTAFLASH™ Needle Technology available from Becton, Dickinson & Company of Franklin Lakes, N.J. In some embodiments, the IV catheter assembly 200 may be similar to the BD SAF-T-INTIMA™ Closed IV Catheter System or the BD NEXIVA™ Closed IV Catheter System in terms of one or more components and/or operation.

In some embodiments, the IV catheter assembly 200 may include a catheter adapter 202, which may include a distal end 204, a proximal end 206, a lumen 208 extending through the distal end 204 of the catheter adapter 202 and the proximal end 206 of the catheter adapter 202, and an inner wall 210 forming the lumen 208. In some embodiments, the IV catheter assembly 200 may include a catheter 212 extending from the distal end 204 of the catheter adapter 202. In some embodiments, the catheter 212 may include a peripheral IV catheter, a midline catheter, or a peripherally-inserted central catheter. In some embodiments, the catheter 212 may be hollow and tubular.

In some embodiments, the IV catheter assembly 200 may include an introducer needle 214, which may include a sharp distal tip 216 and an elongated shaft 218 extending from the sharp distal tip 216. In some embodiments, the introducer needle 214 may extend distally from a needle hub 215 coupled to the catheter adapter 202. In some embodiments, the introducer needle 214 may be movable between a ready position in which the sharp distal tip 216 is distal to a distal end of the catheter 212 and a retracted position proximal to the ready position. In some embodiments, when the introducer needle 214 is in the ready position, the IV catheter assembly 200 may be configured and ready for insertion into the patient.

In some embodiments, the elongated shaft 218 may include a flashback notch 220. In some embodiments, the flashback notch 220 may include a distal end 222 and a proximal end 224. In some embodiments, the flashback notch 220 may include a smooth tapered edge 226 extending from the distal end 222 in a direction that increases a depth of the flashback notch 220. In some embodiments, the smooth tapered edge 226 may be formed with the flashback notch 220 is cut out of the elongated shaft 218. In some embodiments, the distal end 222 may correspond to a distal-most surface of the flashback notch 220 and the proximal end 224 may correspond to a proximal-most surface of the flashback notch 220. In some embodiments, the distal end 222 may correspond to a distal-most surface of the smooth tapered edge 226. In some embodiments, the distal end 222 and the proximal end 224 may be aligned with a longitudinal axis of the introducer needle 214.

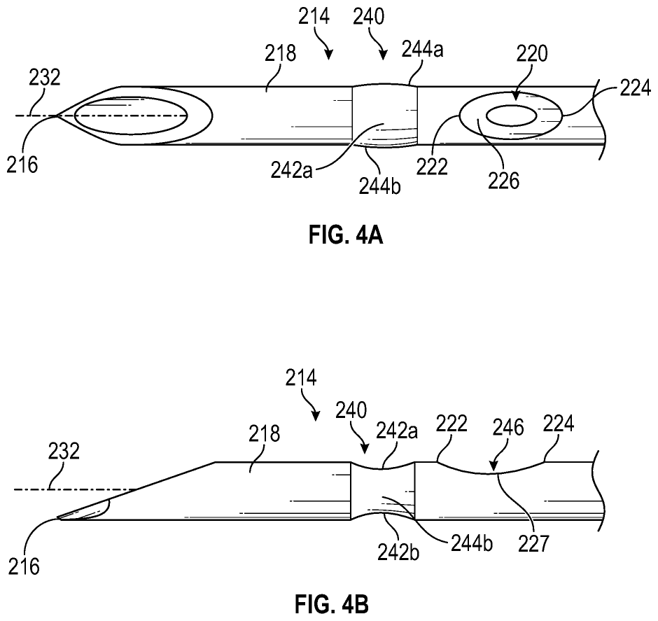

In some embodiments, a proximal end of the smooth tapered edge 226 may be disposed at a deepest point 227 of the flashback notch 220, as illustrated in FIGS. 3B and 3D. In some embodiments, a depth of the flashback notch 220 may increase proximally along an entirety of the smooth tapered edge 226 from the distal end 222. In some embodiments, the smooth tapered edge 226 may extend along at least half of an entire length of the flashback notch 220 between the distal end 222 and the proximal end 224, as illustrated in FIG. 3B (and also FIG. 4B). This may provide for a gradual inclination in a proximal direction from the distal end 222 to decrease a risk of a particular needle guard, such as, for example, the needle guard 120, getting caught on the flashback notch 220 during retraction of the introducer needle 214 in a proximal direction after the catheter 212 is inserted into the vein of the patient.

In some embodiments, the smooth tapered edge 226 extending along at least half of the entire length of the flashback notch 220 may reduce a force needed to retract the introducer needle 214 proximally and overcome the flashback notch 220. As illustrated in FIG. 3A, in some embodiments, a width of the flashback notch 220 may increase from the distal end 222 to the proximal end 224 to form a tear-drop shape. In some embodiments, when the flashback notch 220 has the smooth tapered edge 226, the tear-drop shape may facilitate the flashback notch 220 having a reduced or allowable length without compromising blood flashback through the flashback notch 220.

In some embodiments, the IV catheter assembly 200 may include a needle guard 228 slidably mounted on the introducer needle 214. In some embodiments, the needle guard 228 may be similar or identical to the needle guard 120 of the prior art in terms of one or more components and/or operation. In some embodiments, the needle guard 228 may include any suitable needle guard. Some examples of suitable needle guards may be disclosed in U.S. Pat. No. 7,972,313, filed Nov. 22, 2006, entitled “SPRING CLIP SAFETY IV CATHETER,” which is herein incorporated by reference in its entirety. In some embodiments, a portion of the needle guard 228 may abut the inner wall 210 of the catheter adapter 202, which may include one or more grooves, to retain the needle guard 228 in the catheter adapter 202 when the introducer needle 214 is in the ready position. In some embodiments, in response to movement of the introducer needle 214 from the ready position to the retracted position, the portion of the needle guard 228 may be released from the inner wall and may block the sharp distal tip 216.

In some embodiments, the flashback notch 220 may include a step 230 extending from the proximal end 224 of the flashback notch 220. In some embodiments, the step 230 may be transverse to a longitudinal axis 232 of the introducer needle 214. In some embodiments, the smooth tapered edge 226 may extend adjacent the step 230 as illustrated in FIG. 3D. In other embodiments, the smooth tapered edge 226 may extend to the step 230. In some embodiments, the step 230 that is transverse to the longitudinal axis 232 may allow the smooth tapered edge 226 to be longer while keeping a same length of the flashback notch 220. As illustrated in FIG. 3D, the step 230 may be disposed at an angle a that is equal to 90° with respect to the longitudinal axis 232.

In some embodiments, in an orthogonal side view projection of the IV catheter assembly such as FIG. 3D, every tangent to the smooth tapered edge 226 may be at an angle of 30° or less with respect to the longitudinal axis 232 of the introducer needle 214, which may decrease a risk of a particular needle guard getting caught on the flashback notch 220 during retraction of the introducer needle 214 in the proximal direction after the catheter 212 is inserted into the vein of the patient. FIG. 3D illustrates one example tangent 231 of the smooth tapered edge 226 that is disposed at an angle b with respect to the longitudinal axis 232, according to some embodiments. In some embodiments, to decrease a risk of a particular needle guard getting caught on the flashback notch 220 during retraction, every tangent to the smooth tapered edge 226 may be at an angle with respect to the longitudinal axis 232 that is less than or equal to 60°, less than or equal to 50°, less than or equal to 40°, less than or equal to 30°, or less than or equal to 20°. It is understood that the angle of the smooth tapered edge 226 with respect to the longitudinal axis 232 is greater than 0°. In some embodiments, in the orthogonal side view projection of the IV catheter assembly, every tangent to the smooth tapered edge 226 may be at an angle between 10° and 30, inclusive, with respect to the longitudinal axis 232, which may decrease the risk of the needle guard catching on the smooth tapered edge 226 and also facilitate flash blood flow.

In some embodiments, the smooth tapered edge 226 including a tangent at the distal end 222 that is approximately 30° with respect to the longitudinal axis 232, such as between 25° and 30° or between 30° and 35°, may improve smooth retraction of the needle guard 228 with respect to the introducer needle 214 more than the smooth tapered edge 226 including a tangent that is greater than 30° with respect to the longitudinal axis 232. In some embodiments, when the smooth tapered edge 226 includes a tangent at the distal end 222 that is less than approximately 10° with respect to the longitudinal axis 232, the flashback notch 220 may not be deep enough to facilitate flash blood flow therethrough for visualization.

In some embodiments, the flashback notch 220 may be on a top of the introducer needle 214, which may facilitate visualization of flash blood exiting therefrom. The top of the introducer needle 214 may be directly between the patient and the clinician. In these embodiments, the flashback notch 220 may be aligned with and on a same side of the introducer needle 214 as a bevel of the sharp distal tip 216.

In some embodiments, the introducer needle 214 further may include a needle feature 240 disposed on the elongated shaft 218, which may be cylindrical. In some embodiments, the needle feature 240 may include a first indent 242a and a second indent 242b opposite the first indent 242a. In some embodiments, the first indent 242a may be aligned with the flashback notch 220. In some embodiments, the needle feature 240 may include a first protrusion 244a and a second protrusion 244b opposite the first protrusion 244a. In some embodiments, the first protrusion 244a and the second protrusion 244b may be disposed between the first indent 242a and the second indent 242b.

In some embodiments, the first indent 242a may be disposed on the top of the introducer needle 214, which may be configured to contact the needle guard 228 when the introducer needle 214 is retracted proximally with respect to the needle guard 228. In some embodiments, the second indent 242b may be disposed on a bottom of the introducer needle 214, which may also be configured to contact the needle guard 228 when the introducer needle 214 is retracted proximally with respect to the needle guard 228. Thus, in some embodiments, the first indent 242a and/or the second indent 242b may reduce a risk of the needle guard 228 catching on the needle feature 240.

In some embodiments, the first protrusion 244a and the second protrusion 244b may be disposed on opposing sides of the introducer needle 214. In some embodiments, a proximal end of the introducer needle 214 may pass through an opening in a rear wall of the needle guard 228, but the introducer needle 214 may be stopped from passing entirely through the needle guard 228 due to interference of the first protrusion 244a and/or the second protrusion 244b interfering with the opening, which may have a smaller diameter than the needle feature 240. In some embodiments, in response to movement of the introducer needle 214 from the ready position to the retracted position, the elongated shaft 218 may no longer exert a force on the portion such that the portion is released from the inner wall of the catheter adapter 202 and blocks the sharp distal tip 216.

Referring now to FIGS. 4A-4B, in some embodiments, a flashback notch 246 may have an oval shape. In some embodiments, the flashback notch 246 may be similar or identical to the flashback notch 220 in terms of one or more components and/or operation. In some embodiments, when the flashback notch 246 has the smooth tapered edge 226, the oval shape may facilitate the flashback notch 246 having a reduced or allowable length without compromising blood flashback through the flashback notch 220. As mentioned, in some embodiments, the smooth tapered edge 226 extending along at least half of the entire length of the flashback notch 220 may reduce a force needed to retract the introducer needle 214 proximally and overcome the flashback notch 220. As illustrated in FIGS. 4A-4B, the smooth tapered edge 226 may extend approximately half of the entire length of the flashback notch 220. In some embodiments, in an orthogonal side view projection of the IV catheter assembly such as FIG. 4B, every tangent to the smooth tapered edge 226 may be at an angle of 30° or less, which may provide advantages previously discussed.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. An intravenous catheter assembly, comprising:
a catheter adapter, comprising a distal end, a proximal end, a lumen extending through the distal end of the catheter adapter and the proximal end of the catheter adapter, and an inner wall forming the lumen;
a catheter extending from the distal end of the catheter adapter;
an introducer needle comprising a sharp distal tip and an elongated shaft extending from the sharp distal tip, wherein the introducer needle is movable between a ready position in which the sharp distal tip is distal to a distal end of the catheter and a retracted position proximal to the ready position, wherein the elongated shaft comprises a flashback notch, wherein the flashback notch comprises a distal end and a proximal end, wherein the flashback notch comprises a smooth tapered edge extending from the distal end in a direction that increases a depth of the flashback notch, wherein the smooth tapered edge extends along at least half of an entire length of the flashback notch between the distal end and the proximal end; and
a needle guard slidably mounted on the introducer needle, wherein a portion of the needle guard abuts the inner wall of the catheter adapter to retain the needle guard in the catheter adapter when the introducer needle is in the ready position, wherein in response to movement of the introducer needle from the ready position to the retracted position, the portion of the needle guard is released from the inner wall and blocks the sharp distal tip.

2. The intravenous catheter assembly of claim 1, wherein a width of the flashback notch increases from the distal end to the proximal end to form a tear-drop shape.

3. The intravenous catheter assembly of claim 2, wherein the flashback notch comprises a step extending from the proximal end of the flashback notch, wherein the step is transverse to a longitudinal axis of the introducer needle.

4. The intravenous catheter assembly of claim 3, wherein the smooth tapered edge extends adjacent the step.

5. The intravenous catheter assembly of claim 1, wherein the flashback notch has an oval shape.

6. The intravenous catheter assembly of claim 1, wherein in an orthogonal side view projection of the intravenous catheter assembly, every tangent to the smooth tapered edge is at an angle of 30° or less with respect to a longitudinal axis of the introducer needle.

7. The intravenous catheter assembly of claim 1, wherein the introducer needle further comprises a needle feature disposed on the elongated shaft, wherein the needle feature comprises a first indent and a second indent opposite the first indent, wherein the first indent is aligned with the flashback notch.

8. The intravenous catheter assembly of claim 7, wherein the needle feature comprises a first protrusion and a second protrusion opposite the first protrusion, wherein the first protrusion and the second protrusion are disposed between the first indent and the second indent.

9. The intravenous catheter assembly of claim 8, wherein the first indent is disposed on a top of the introducer needle, wherein the second indent is disposed on a bottom of the introducer needle, and wherein the first protrusion and the second protrusion are disposed on opposing sides of the introducer needle.

10. The intravenous catheter assembly of claim 1, wherein in response to movement of the introducer needle from the ready position to the retracted position, the elongated shaft no longer exerts a force on the portion such that the portion is released from the inner wall and blocks the sharp distal tip.

11. An intravenous catheter assembly, comprising:
a catheter adapter, comprising a distal end, a proximal end, a lumen extending through the distal end of the catheter adapter and the proximal end of the catheter adapter, and an inner wall forming the lumen;
a catheter extending from the distal end of the catheter adapter;
an introducer needle comprising a sharp distal tip and an elongated shaft extending from the sharp distal tip, wherein the introducer needle is movable between a ready position in which the sharp distal tip is distal to a distal end of the catheter and a retracted position proximal to the ready position, wherein the elongated shaft comprises a flashback notch, wherein the flashback notch comprises a distal end and a proximal end, wherein the flashback notch comprises a smooth tapered edge extending from the distal end in a direction that increases a depth of the flashback notch, wherein in an orthogonal side view projection of the intravenous catheter assembly, every tangent to the smooth tapered edge is at an angle of 30° or less with respect to a longitudinal axis of the introducer needle; and
a needle guard slidably mounted on the introducer needle, wherein a portion of the needle guard abuts the inner wall of the catheter adapter to retain the needle guard in the catheter adapter when the introducer needle is in the ready position, wherein in response to movement of the introducer needle from the ready position to the retracted position, the portion of the needle guard is released from the inner wall and blocks the sharp distal tip.

12. The intravenous catheter assembly of claim 11, wherein a width of the flashback notch increases from the distal end to the proximal end to form a tear-drop shape.

13. The intravenous catheter assembly of claim 12, wherein the flashback notch comprises a step extending from the proximal end of the flashback notch, wherein the step is transverse to a longitudinal axis of the introducer needle.

14. The intravenous catheter assembly of claim 13, wherein the smooth tapered edge extends adjacent the step.

15. The intravenous catheter assembly of claim 11, wherein the flashback notch has an oval shape.

16. The intravenous catheter assembly of claim 11, wherein the introducer needle further comprises a needle feature disposed on the elongated shaft, wherein the needle feature comprises a first indent and a second indent opposite the first indent, wherein the first indent is aligned with the flashback notch.

17. The intravenous catheter assembly of claim 16, wherein the needle feature comprises a first protrusion and a second protrusion opposite the first protrusion, wherein the first protrusion and the second protrusion are disposed between the first indent and the second indent.

18. The intravenous catheter assembly of claim 17, wherein the first indent is disposed on a top of the introducer needle, wherein the second indent is disposed on a bottom of the introducer needle, and wherein the first protrusion and the second protrusion are disposed on opposing sides of the introducer needle.

19. The intravenous catheter assembly of claim 11, wherein the smooth tapered edge extends along at least half of an entire length of the flashback notch between the distal end and the proximal end.

20. An introducer needle, comprising:
a sharp distal tip; and
an elongated shaft extending from the sharp distal tip, wherein the elongated shaft comprises a flashback notch, wherein the flashback notch comprises a distal end and a proximal end, wherein the flashback notch comprises a smooth tapered edge extending from the distal end in a direction that increases a depth of the flashback notch, wherein a width of the flashback notch increases from the distal end to the proximal end to form a tear-drop shape.
